(19) United States
(12) Patent Application Publication
(10) Pub. No.: US 2014/0303547 A1
(43) Pub. Date: Oct. 9, 2014

(54) PHOTOTHERAPEUTIC DEVICE, METHOD AND USE

(71) Applicant: KLOX TECHNOLOGIES INC., LAVAL (CA)

(72) Inventors: Nikolaos Loupis, Athens (GR); Remigio Piergallini, Grottammare Ascoli Piceno (IT)

(73) Assignee: KLOX TECHNOLOGIES INC., LAVAL (CA)

(21) Appl. No.: 14/194,216

(22) Filed: Feb. 28, 2014

Related U.S. Application Data
(60) Provisional application No. 61/786,140, filed on Mar. 14, 2013.
(60) Provisional application No. 61/771,466, filed on Mar. 1, 2013.

(51) Int. Cl. A61N 5/06; H05B 33/08; A61K 41/00

(52) U.S. Cl. CPC A61N 5/062; A61K 41/0057; A61N 5/0624; H05B 33/0845; H05B 33/0857; A61N 2005/0663; A61N 2005/0626
USPC 604/20; 607/88; 607/90; 315/192

(57) ABSTRACT

Disclosed herein are devices, systems, and methods for providing light therapy to a subject's tissues. The devices, systems, and methods include a phototherapeutic lamp comprising light generating sources that emit light capable of causing a medical and/or cosmetic treatment of tissues. Also included are uses of the devices. The devices and methods may also include a photoactivatable composition.

### CROSS-REFERENCE TO RELATED APPLICATIONS

This application claims the benefit of U.S. Provisional Application Ser. No. 61/771,466, filed Mar. 1, 2013, and U.S. Provisional Application Ser. No. 61/786,140, filed Mar. 14, 2013, which applications are incorporated herein by reference.

### BACKGROUND OF THE DISCLOSURE

Phototherapy relates to treatment of biological tissues using electromagnetic radiation such as visible and infrared lights. It has a wide range of applications in both the medical and cosmetic fields including skin rejuvenation and treatment of various skin conditions. Phototherapy has also been used in combination with certain photo-sensitive drugs or photoactive compositions. It is desired to provide a novel phototherapy device and method having cosmetic or medical uses.

### SUMMARY

There is broadly provided a device for phototherapy, and a method and use of the device and which comprises light having different and complementary therapeutic effects to treat a variety of different conditions in a subject, which may be human or animal.

Different aspects of the device broadly comprise at least one light source which can emit light having an emission spectra which can have one or more of the following properties: (i) produce an antimicrobial effect on a treatment area irradiated with the emitted light, (ii) modulate blood flow in the treatment area, and/or (iii) activate a photoactivatable composition comprising a chromophore/fluorochrome which may then emit a light (fluorescence or phosphorescence) with therapeutic properties such as modulating blood flow in the treatment area, collagen modulation and/or which can release bio-therapeutic reactive species, such as singlet oxygen, onto, into or nearby the treatment area.

From one aspect, there is provided a device for phototherapy comprising: a first light source which can emit a first light having an emission spectra for activating a photoactivatable composition applied on or near a treatment area; and a second light source which can emit a second light having a different emission spectra from the first, wherein the first and the second emission spectra are in the blue and/or violet regions of the electromagnetic spectrum. In certain embodiments, the first light is in the violet region of the electromagnetic spectrum and the second light is in the blue region of the electromagnetic spectrum.

In certain embodiments, the first light has a peak emission wavelength of about 430 to about 500 nm, about 440 to about 500 nm, about 450 to about 500 nm, about 430 to about 475 nm, about 435 nm to about 470 nm, about 440 nm, about 450 nm, about 460 nm or about 470 nm. In certain embodiments, the second light has a peak emission wavelength of about 400 nm to about 500 nm, about 400 nm to about 475 nm, about 400 nm to about 450 nm, about 400 nm to about 430 nm, or about 410 nm to about 420 nm, or about 415 nm. At least one of the first and second lights can have a bandwidth (full width half maximum) of equal to or less than about 20 nm, or about 19 nm±5 nm (14 nm to 24 nm).

In certain embodiments, an average power density of the light emitted by the device, measured at a treatment distance (such as 5 cm or 10 cm), is less than about 200 mW/cm², is about 10 to about 200 mW/cm², about 10 to about 150 mW/cm², 20 to about 130 mW/cm², about 55 to about 130 mW/cm², about 90 to about 140 mW/cm², about 100 to about 140 mW/cm², or about 110 to about 135 mW/cm². In some embodiments, an average power density of the light emitted by the device is about 10 to about 75 mW/cm², about 30 to about 70 mW/cm², about 40 mW/cm² to about 70 mW/cm², or about 55 to about 65 mW/cm².

The device is arranged to emit light having a fluence, during a single treatment, of more than about 4 J/cm², more than about 10 J/cm², more than about 15 J/cm², more than about 30 J/cm², more than about 50 J/cm², up to about 60 J/cm². In certain embodiments, the device is arranged to emit light having a fluence, during a single treatment, of about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², or about 10 J/cm² to about 20 J/cm². The treatment time may range from about 30 seconds to about 25 minutes, typically 5 to 15 minutes. The maximum light intensity can be about 12 J/cm² per minute of treatment.

In some embodiments, the peak emission wavelength of the first light is from about 440 nm to about 470 nm and has a bandwidth of about 18-24, and the peak emission wavelength of the second light is from about 410 nm to about 430 nm and has a bandwidth of about 13-18 nm. In those embodiments, the device can emit an average power density of about 55 to about 130 mW/cm², or about 10 to about 75 mW/cm².

In certain embodiments, the device further includes a controller, which may be in electronic communication with the light sources for varying one or more of emission spectra parameters of the first and second lights, the emission spectra parameters being selected from bandwidth, peak wavelength, power density, time of emission and fluence. The controller may be able to control separately one or more of the emission spectra parameters of the first and second lights. In some embodiments, the controller is arranged to modulate one or more of the emission spectra parameters of the first and second lights as a function of treatment time. In some embodiments, the controller may be arranged to modulate the emitted power density levels of the first and second light sources as a function of treatment time, for example, by diminishing the emitted power density of the first light source during the light emission time, and optionally by increasing the emitted power density of the second light source during the light emission time, to mimic a fluorescence or a phosphorescence emitted by activated chromophores. The controller may include treatment modes with pre-set treatment parameters including emitted light density, wavelength, variation of emitted light density and wavelength with time, and treatment distance. For example, the treatment modes may include different treatment parameters for mild acne, severe acne, deep wrinkles, mild wrinkles, different grades of ulcers. In certain embodiments, the controller can control a pulsing of any of the light sources.

In certain embodiments, the device further comprises a third light source, wherein the third light source can emit a third light having a peak wavelength of about 500 nm to about 750 nm, about 630 to about 750 nm.

In certain embodiments, the first light source and the second light source (when present), can emit non-coherent light. For example, the first light source and the second light source (when present), can be LEDs. The LEDs can be arranged as an array. The LEDs from the first light source may be considered as a set of LEDs, and the LEDs from the second light source may be considered as another set of LEDs, each set of LEDs comprising at least one LED. The LEDs from each set may be arranged in an interdispersed fashion or in a side-by-side fashion. The array of LEDs may be arranged on at least one panel. The device may comprise a plurality of connectable panels to present a flat or curved light emitting surface. The connectable panels may be moveable with respect to each other or in a fixed configuration. The device may also include a heatsink coupled to the array of LEDs, and a fan for cooling the array of LEDs.

In another embodiment, the array of LEDs may be provided on a flexible substrate such as a fabric, for use with a dressing or a mask, or forming part of a dressing or a mask.

The light emitting surface of the device may comprise one or more waveguides such as a fibre optic, or a bundle of fibre optics connectable to the one or more light sources. The fibre optics may be made of any material with suitable light carrying and tensile properties, such as polymethylmethacrylate (PMMA). The fibre optic(s) may be encased in a sleeve. The fibre optics can thus be used to deliver the therapeutic light from the device to an internal cavity of a subject or a hard to reach treatment area on the subject.

From another aspect, there is provided a device for phototherapy comprising: a first light source which can emit a first light having a peak emission wavelength of about 400 to about 750 nm, and a power density of about 10 to about 75 mW/cm², or about 55 mW/cm² to about 150 mW/cm².

From a further aspect, there is provided a device for phototherapy comprising: a first light source which can emit a first light having a peak emission wavelength of about 400 to about 750 nm, and a bandwidth of about 19 nm±about 5 nm.

From another aspect, there is provided a device for phototherapy comprising: a first light source which can emit a first light having a peak emission wavelength of about 400 to about 750 nm, and a fluence during a single treatment of about 4 to about 60 J/cm², about 10 to about 60 J/cm², about 10 to about 50 J/cm², about 10 to about 40 J/cm², about 10 to about 30 J/cm², about 20 to about 40 J/cm², or about 10 to about 20 J/cm².

From a yet further aspect, there is provided a device for phototherapy having at least one light source arranged to emit light having a bandwidth of more than about 15 nm, having a peak wavelength of between about 400 nm to about 700 nm, and wherein the emitted power density of the light is decreased over a light emission period. The power density may be decreased at any rate, for example at about 0.002 mW/cm² per minute of irradiation to about 0.1 mW/cm² per minute of irradiation, about 0.005 mW/cm² per minute, about 0.006 mW/cm² per minute, or about 0.012 mW/cm² per minute. This may substantially simulate a fluorescence emitted by a chromophore and the decay of the power intensity of the fluorescence with time.

The disclosure contemplates that any of the embodiments set forth below can be combined with each other or with any of the aspects or embodiments set forth above, or otherwise set forth herein.

In certain embodiments of the above aspects, the light source can emit light within the violet range (400-450 nm), the blue range (450-490 nm), the green range of the electromagnetic spectrum (about 490 to about 560 nm), the yellow range (560-590 nm), the orange range (590-635), or the red range of the electromagnetic spectrum (about 635 to about 750 nm). These emitted wavelengths together with the power density, fluence and/or bandwidths described above, can photoactivate biophotonic compositions, and/or have a therapeutic effect themselves.

In certain embodiments, the peak emission wavelength emitted by the first light source is about 440-470 nm, has a bandwidth of about 20 nm±2 nm, and a maximum power density at 5 cm of between about 100-150 mW/cm², 60-135 mW/cm², about 135 mW/cm². In another embodiment, the peak emission wavelength emitted by the first light source is about 440-480 nm, and has a maximum power density at 5 cm or at 10 cm of less than about 75 mW/cm², about 25 to about 70 mW/cm², about 30 to about 65 mW/cm², about 55 to about 65 mW/cm². In another embodiment, the device includes a second light source which can emit light having a peak wavelength of about 410 nm to about 420 nm, a bandwidth of about 13-15 nm and a maximum power density within the range 0.01 to 5 mW/cm².

At least one light source having a wavelength within the violet/blue ranges with a peak of about 440-470 nm has been found to excite yellow/orange/red dyes, such as Eosin Y, Fluorescein, Rose Bengal, Phloxine B, and Erythrosine, each of which, together with the violet/blue light, has been observed by the inventors to have beneficial effects at the treatment area such as modulation of blood flow and collagen modulation.

In certain embodiments, the light emitted from the light source, as well as being an activating light for a chromophore in a photoactivatable composition, may also have therapeutic benefits itself when applied to tissue e.g. antimicrobial properties, modulation of blood flow at the treatment site, collagen modulation, or any other cosmetic or medical therapeutic effects. Therefore, it may be advantageous to use the device with a photoactivatable composition which also allows the activating light to pass therethrough in order to be able to irradiate the tissues onto which the composition is applied. The photoactivatable composition may be substantially transparent or translucent, or otherwise optically conductive.

The first, second and/or third light sources may emit light having different/complementary properties simultaneously, at different times and/or for different time periods, from a single light source or a plurality of light sources. In this way, the device may be used to treat different stages of a condition for example treating an infection on a wound first, followed by a reduction in inflammation, and collagen modulation to minimize scarring during wound healing. Another example is to treat acne by initially killing the bacteria thought to be responsible for the condition (e.g. propionibacterium acnes (p. acnes)) on the skin of subject, followed by vascularization and collagen modulation to heal the acne lesions and scars.

The device of any of the above embodiments may be a head for a lamp, or the lamp itself. When the device is a head, it may be interchangeable with other lamp heads and configured to fit on the same lamp base structure. The light sources on different heads may be configured to emit different parameters, such as wavelength, pulse duration, total emitted energy, or the different heads may have different sizes and shapes suitable for treatment of different body parts.

From a yet further aspect, there is provided a lamp, comprising a lamp head having a plurality of light emitting diodes (LEDs) arranged in an array, the array comprising at least two sets of LEDs, wherein each set includes at least one LED; a lamp controller electrically connected to the lamp head and having circuitry for controlling and operating the LEDs; wherein the first set of LEDs can generate non-coherent light having a peak wavelength of about 430 nm to about 500 nm; wherein the second set of LEDs can generate non-coherent light having a peak wavelength of about 400 nm to about 430 nm; and wherein a power density of light which can be generated by the lamp head is from about 10 to about 75 mW/cm², or from about 55 mW/cm² to about 150 mW/cm². The first set of LEDs can generate light having a full width half maximum bandwidth of about 13 to about 26 nm, and the second set of LEDs can generate light having a full width half maximum bandwidth of about 13 nm to about 20 nm. The lamp may further comprise a third set of LEDs, wherein the third set of LEDs can generate non-coherent light having a peak wavelength of about 500 nm to 750 nm, or about 630 to about 720 nm.

In some embodiments, the device or the lamp is portable. It may be provided with wheels or handle(s). The device or lamp may include a mount for mounting the lamp to furniture, such as a bed, or to a wall. The device or lamp may also include a support to support the device or lamp on a floor such as legs, feet, wheels, base. The support may include a weighted base or a long foot which can slide under furniture and prevent the device or lamp from falling over. The mount or the support may be foldable for ease of storage and portability. A portable version of the device or the lamp may be battery operated or rechargeable, and may be provided with or without a cable.

In one embodiment of a portable hand-held lamp, the lamp head has a circular emitting surface of a diameter of, for example, about 5 cm to about 10 cm. In this case, in order to treat a larger diameter treatment area than the beam diameter, LEDs with a broad divergence angle are used, and the portable lamp is held at a sufficient distance from the treatment area in order to provide light over a larger diameter than the diameter of the emitting surface. In one embodiment, the light density is about 75 to about 120 mW/cm². The wavelength is about 420-490 nm with a peak around 460-470 nm.

From another aspect, there is provided a device having at least one light source which can emit light substantially corresponding to a fluorescence or a phosphorescence emitted by an activated chromophore. In one embodiment, the light source is arranged to emit light having a bandwidth of more than about 15 nm, more than about 20 nm, more than about 25 nm, more than about 30 nm. The bandwidth may be for example about 15 nm to about 100 nm, about 25 nm to about 80 nm, about 30 nm to about 70 nm, or about 20 nm to about 50 nm. In certain embodiments, the device is arranged to modulate the emitted light source over a period of light emission from the device, for example a decrease in emitted power density over time. In certain embodiments, the at least one light source is arranged to emit light having a peak wavelength of between about 400 nm to about 750 nm, about 480 nm to about 700 nm, about 500 nm to about 660 nm, about 540 nm to about 640 nm. In one embodiment, the light source can generate light with a maximum power density of between 0.005 to about 10 mW/cm², about 0.01 to 0.1 mW/cm², about 0.01 to about 2 mW/cm², about 0.01 to about 3 mW/cm², about 0.5 to about 5 mW/cm².

The power density may be decreased at any rate, for example at about 0.002 mW/cm² per minute of irradiation to about 0.1 mW/cm² per minute of irradiation, about 0.005 mW/cm² per minute, about 0.006 mW/cm² per minute, or about 0.012 mW/cm² per minute.

From another aspect, there is provided use of a device or a lamp as defined above with a photoactivatable composition. In certain embodiments, the photoactivatable composition includes at least one of a green, yellow, orange or red photoactive agent. In one implementation, the photoactive agent is within an optical medium which can be applied to the tissue or placed near the tissue and has a usual peak excitation wavelength (in water or alcohol) in the range of about 400 to about 700 nm, about 420-565 nm, about 420-540 nm, 470-535 nm. In certain embodiments, the photoactivatable composition comprises any one or more of Eosin Y, Fluorescein, Rose Bengal, Erythrosine, Phloxine B, chlorophyll a, chlorophyll b, chlorophilin.

From a further aspect, there is provided use of a device or a lamp as described above for cosmetic use (e.g. skin rejuvenation, skin conditioning, skin maintenance, reducing or eliminating scarring, removing tattoos, evening skin tone), medical use (e.g. wound healing, treating inflammation, treating bacterial, viral or fungal infections, treating skin conditions such acne, rosacea, psoriasis, dermatitis) and/or diagnostic use. The device and/or lamp may be used in any setting including at home, hospital, clinic, field etc.

In certain embodiments of the uses above, the device can be a mobile device such as a hand-held computing device or a mobile telephone with a display screen or a flashlight as the emitting surface, e.g. Iphone®, Ipad®, Samsung Galaxy®. In these embodiments, ‘apps’ which emit light having emission spectra which can activate a photoactivatable composition can be used. In the same way, a display screen of a desktop computer or a television can be adapted to emit light having emission spectra which can activate a photoactivatable composition. In this way, a user may benefit from a therapeutic effect of light and a photoactivatable composition simply by positioning the area of treatment near a light emitting surface of the mobile device or display screen.

From a yet further aspect, there is provided a cosmetic method or a medical method for treating tissues, said method comprising: irradiating a treatment area with light having an emission spectra as defined above (in relation to the light emitted by the devices and lamps of the present disclosure). For example, the treatment area may be irradiated with two lights which have different emission spectra within the blue and/or violet regions of the electromagnetic spectrum.

The method may comprise irradiating the tissue with a first light having a peak emission wavelength of about 400 to about 750 nm, and modulating at least one of the peak emission wavelength, bandwidth, power density or fluence of the first light during the irradiation of the tissue. In one implementation, the method comprises decreasing or increasing the maximum power intensity of the light emitted from at least one light source during the time of light irradiation. Lights from different light sources may be modulated differently, at different times or at the same time. It will be understood that that the modulation of light from one light source may occur over only a portion of the total irradiation time, or over the full irradiation time. In certain embodiments, the power density may be increased or decreased at a rate, for example, of at about 0.002 mW/cm² per minute of irradiation to about 0.1 mW/cm² per minute of irradiation, about 0.005 mW/cm² per minute, about 0.006 mW/cm² per minute, or about 0.012 mW/cm² per minute.

The method may comprise irradiating the tissue with a first light having a peak emission wavelength of about 400 to about 750 nm, and a power density of about 10 to about 75 mW/cm², or about 55 mW/cm² to about 150 mW/cm².

The method may comprise irradiating the tissue with a first light having a peak emission wavelength of about 400 to about 750 nm, and a bandwidth of about 19 nm±about 5 nm.

The method may comprise irradiating the tissue with a first light having a peak emission wavelength of about 400 to about 750 nm, and a fluence during a single treatment of about 4 to about 60 J/cm², about 10 to about 60 J/cm², about 10 to about 50 J/cm², about 10 to about 40 J/cm², about 10 to about 30 J/cm², about 20 to about 40 J/cm², or about 10 to about 20 J/cm². The treatment area may be irradiated simultaneously or at different times, from a single light source or a plurality of light sources with light having different properties. The irradiating light may have any of the properties described above in relation to aspects of the device and lamp.

The treatment time may range from about 30 seconds to about 30 minutes, typically 5 to 15 minutes. The maximum light intensity can be about 12 J/cm² per minute of treatment. The light may be applied continuously or pulsed.

In certain embodiments, the irradiating light is a fluorescence or phosphorescence light within one or more of the green, yellow, orange, red and infrared portions of the electromagnetic spectrum, for example having a peak wavelength within the range of about 490 nm to about 720 nm. In one embodiment, the irradiating light has a wavelength of between about 400 nm to about 700 nm, about 480 nm to about 700 nm, about 500 nm to about 660 nm, about 540 nm to about 640 nm. In another embodiment, the irradiating light has a power density of between 0.005 to about 10 mW/cm², about 0.5 to about 5 mW/cm². In certain embodiments, the irradiating light has a bandwidth of about 15 nm to about 100 nm, about 25 nm to about 80 nm, about 30 nm to about 70 nm, or about 20 nm to about 50 nm. The light source of the irradiating light may be a photoactive agent such as a fluorochrome which is activated by the first light source, or any other light source, to emit fluorescence. Alternatively, the irradiating light may be from an electronically generated light such as LED, laser etc which mimics a fluorescence or phosphorescence spectra.

In certain embodiments, the maximum power density of the irradiating light is from about 0.01 mW/cm² to about 200 mW/cm², 0.02 mW/cm² to about 150 mW/cm², 0.02 mW/cm² to about 135 mW/cm², 0.02 mW/cm² to about 75 mW/cm², 0.02 mW/cm² to about 60 mW/cm², about 0.02 mW/cm² to about 50 mW/cm², about 0.02 mW/cm² to about 30 mW/cm², about 0.02 mW/cm² to about 15 mW/cm².

It will be clear to a skilled person that combinations of the embodiments of the irradiating light described above are also possible. For example, irradiating the treatment area with light having different emission spectra for the same or different times. In one embodiment, the treatment area is irradiated with light from a first light source having a peak emission wavelength of about 440-470 nm, a bandwidth of about 20 nm±2 nm, and a maximum power density between about 60-150 mW/cm²; and light from a second source having a peak emission wavelength of about 540 nm to about 640 nm, a power density of between 0.005 to about 10 mW/cm², about 0.5 to about 5 mW/cm², and a bandwidth of about 20 nm to about 100 nm, about 25 nm to about 80 nm, about 30 nm to about 70 nm, or about 20 nm to about 50 nm.

From another aspect, there is provided a system for treating tissues, said system comprising: at least one light source for irradiating a treatment area, the at least one light source being able to emit irradiating light having an emission spectra which can have one or more of the following properties: (i) produce an antimicrobial effect on a treatment area irradiated with the emitted light, (ii) modulate blood flow in the treatment area, and/or (iii) activate a photoactivatable composition comprising a chromophore which may then emit a light with therapeutic properties or which can release bio-therapeutic reactive species onto, into or nearby the treatment area such that a photoactive agent in the photoactivatable composition is not internalized by the cells and/or does not sensitize the cells. The treatment area may be irradiated simultaneously or at different times, from a single light source or a plurality of light sources, with the light having the different properties. The irradiating light may have any of the properties described above in relation to aspects of the device, lamp and/or system.

In certain embodiments, the emitted light from the at least one light source has a peak emission wavelength of about 400 nm to about 720 nm, 400 nm to about 550 nm, about 450 nm to about 500 nm, about 440 to about 475 nm, about 450, about 446, about 464 nm or about 470 nm. The light source in this case can be at least one LED or any array of LEDs.

In certain embodiments, the irradiating light is fluorescence or phosphorescence light within one or more of the green, yellow, orange, red and infrared portions of the electromagnetic spectrum, for example having a peak wavelength within the range of about 520 nm to about 720 nm. In this case, the system includes a photoactive agent as a light source. In one embodiment, the irradiating light has a wavelength of between about 400 nm to about 700 nm, about 480 nm to about 700 nm, about 500 nm to about 660 nm, about 540 nm to about 640 nm. In another embodiment, the irradiating light has a power density of between 0.005 to about 10 mW/cm², about 0.5 to about 5 mW/cm². In certain embodiments, the irradiating light has a bandwidth of about 20 nm to about 100 nm, about 25 nm to about 80 nm, about 30 nm to about 70 nm, or about 20 nm to about 50 nm. The light source of the irradiating light may be a photoactive agent such as a fluorochrome which is activated by the first light source, or any other light source, to emit fluorescence. Alternatively, the light source may be from an array of LEDs which substantially mimics a fluorescence or phosphorescence spectra.

In certain embodiments, the light source can generate light having a peak emission wavelength of about 400 nm to about 500 nm, optionally about 400 nm to about 475 nm, optionally about 400 nm to about 450 nm, optionally about 410 nm to about 420 nm, or in a certain embodiment about 415 nm to about 418 nm. The light source in this case can be at least one LED or any array of LEDs.

It will be clear to a skilled person that combinations of one or more of the embodiments of the light sources described above are also possible within the system of the disclosure. For example, the system may comprise a first light source having a peak emission wavelength of about 450 nm, a bandwidth of about 20 nm or less, and a maximum power density between about 130-150 mW/cm²; and a second source which is a photoactivatable composition which can emit fluorescence or phosphorescence. In one embodiment, the fluorescence has a peak emission wavelength of about 540 nm to about 640 nm, a power density of between 0.005 to about 10 mW/cm², about 0.5 to about 5 mW/cm², and a bandwidth of about 20 nm to about 100 nm, about 25 nm to about 80 nm, about 30 nm to about 70 nm, or about 20 nm to about 50 nm.

In certain embodiments, the system comprises a controller for modulating the irradiating light during the treatment period, for example, by modulating the emitted maximum power density of the irradiating light over a treatment time or by modulating a bandwidth or wavelength range. In one implementation, the method comprises decreasing or increasing the maximum power intensity of the light emitted from at least one light source during the time of light irradiation. Lights from different light sources may be modulated differently, at different times or at the same time. The power density of any of the emitted lights may be decreased at any rate, for example at about 0.002 mW/cm² per minute of irradiation to about 0.1 mW/cm² per minute of irradiation, about 0.005 mW/cm² per minute, about 0.006 mW/cm² per minute, or about 0.012 mW/cm² per minute. It will be understood that that the power density modulation may occur over only a portion of the total irradiation time, or over the full irradiation time.

From another aspect, there is provided a cosmetic or a medical method of treating tissue, said method comprising irradiating a treatment site of a tissue with a first light which decreases in power density during at least a portion of the total irradiation time. Optionally, the method can comprise irradiating the same treatment site of the tissue with a second light which increases, stays the same or decreases in power intensity during at least a portion of the total irradiation time. In one embodiment, the first light has a multi-colour bandwidth and comprises one or more of a green, yellow, orange or red light. In one embodiment, the second light has a wavelength range within a single colour and comprises a blue or violet light. In certain embodiments, the wavelength and/or bandwidth of the first light and/or second light also changes during at least a portion of the total irradiation time. In other embodiments, the first light is generated by at least one fluorochrome in a composition on or near the treatment site. The method may further comprise further step(s) of modulating the power density of the first light or the second light by varying a thickness of the composition, by varying the distance of a light source from the treatment site, by varying a concentration of the fluorochrome in the composition, or by adding chemical species to the fluorochrome to enhance fluorescence such as halides.

From a yet further aspect, there is provided a system for treating tissues, said system comprising a first light source which can generate a first light which can decrease in power density during at least a portion of the total irradiation time. Optionally, the system comprises a second light source which can generate a second light which increases, stays the same or decreases in power intensity during at least a portion of the total irradiation time. In one embodiment, the first light has a multi-colour bandwidth and comprises one or more of a green, yellow, orange or red light. In one embodiment, the second light has a wavelength range within one colour and comprises a blue or violet light. In certain embodiments, the wavelength and/or bandwidth of the first light and/or second light also changes during at least a portion of the total irradiation time. In other embodiments, the first light source comprises at least one fluorochrome in a composition on or near the treatment site.

Without being bound to theory, the individual and combined wavelengths, bandwidths, emitted power densities and intensities described above are thought to have certain complementary therapeutic effects on tissues which they irradiate. The inventors have observed that light with a peak wavelength of about 410-490 nm has blood flow modulation properties at the treatment site and may have anti-inflammatory and collagen modulation properties. It has also been discovered by the inventors that blue light with a peak wavelength of about 410-490 nm and a bandwidth of about 15-25 nm can photoactivate green, yellow and orange dyes in an optical medium on or near the tissues causing them to fluoresce. According to Stokes' shift the emitted fluorescent light has a longer peak wavelength than the activating light, the longer wavelengths having deeper tissue penetration. The emitted fluorescent light can be multi-colour bandwidth which is thought to enhance its potentially therapeutic effect. Therefore, it is believed that the emitted fluorescent light also has a therapeutic effect on tissues which it irradiates, and which is complementary to the therapeutic effect of the light emitted from the light source. In the presence of oxygen, the photoactivation may also generate reactive oxygen species at levels having a therapeutic effect.

### DEFINITIONS

As used in this specification and the appended claims, the singular form “a”, “an” and “the” include plural referents unless the context clearly dictates otherwise.

As used herein, the term “about” in the context of a given value or range refers to a value or range that is within 20%, preferably within 10%, and more preferably within 5% of the given value or range.

It is convenient to point out here that “and/or” where used herein is to be taken as specific disclosure of each of the two specified features or components with or without the other. For example “A and/or B” is to be taken as specific disclosure of each of (i) A, (ii) B and (iii) A and B, just as if each is set out individually herein.

By “antimicrobial effect” is meant that microorganisms such as bacteria, fungi and protozoans can be killed or inhibited.

By “emission spectra” is meant the spectrum of frequencies of electromagnetic radiation defining the properties of the emitted light, usually in terms of wavelength, power density, and bandwidth.

By “light emitting diode” is meant any light emitting diode including organic, polymer, solid-state and RGB.

By “light source” is meant any source of light which can output light having the desired characteristics, such as light emitting diodes; incandescent light bulbs; electron stimulated; electroluminescent materials such as electroluminescent wires and sheets, field-induced polymer electroluminescent materials; gas discharge bulbs such as fluorescent lamps, cathode lamps, neon and argon lamps, plasma lamps, xenon flash lamps; high intensity discharge lamps such as metal-halide lamps, diode lasers, fiber lasers, arc discharge or other light sources. The light source can emit a pulsed or continuous light wave which may be spectrally concentrated or spectrally diffuse (i.e., broadband). A photoactive agent or agents which can emit light is also considered a light source herein. A light source can be understood to also include a set of one or more light generating units having similar properties e.g. similar wavelengths.

By “photoactivatable composition” is meant any medium including a chromophore in which the molecules of the chromophore are able to absorb radiant energy within the medium leading to the emission of absorbed light, for example as fluorescence, or transition of the chromophore molecules to an excited state and subsequent interaction with other molecules. The excited state is referred to herein, interchangeably, as ‘photoexcited’ or ‘photoactivated’.

Terms “chromophore”, “photoactivating agent” and “photoactivator” are used herein interchangeably. A chromophore means a chemical molecule or compound, when contacted by light irradiation, is capable of absorbing the light.

Further areas of applicability of the disclosed devices, lamps, uses and methods will become apparent from the detailed description provided hereinafter. It should be understood that the detailed description and specific examples, while indicating particular embodiments, are intended for purposes of illustration only and are not intended to limit the scope of the disclosure or any claims that may be pursued.

### BRIEF DESCRIPTION OF THE FIGURES

The foregoing and other objects and advantages will be appreciated more fully from the following further description thereof, with reference to the accompanying drawings. These depicted embodiments are to be understood as illustrative and not as limiting in any way.

FIG. 1 shows a schematic view of a device for emitting light comprising first and second light sources, according to certain embodiments of the present disclosure.

FIG. 2 shows an exemplary physical arrangement of the array of light generating sources of the device of FIG. 1, according to certain embodiments of the present disclosure.

FIG. 3 is a spectrum of the light emitted by the device of FIG. 1, according to certain embodiments of the present disclosure, when measured at 5 cm from the light source.

FIG. 4A shows a first exemplary physical arrangement of arrays of light generating sources.

FIG. 4B shows a second exemplary physical arrangement of arrays of light generating sources.

FIG. 4C shows a third exemplary physical arrangement of arrays of light generating sources.

FIG. 4D shows a fourth exemplary physical arrangement of arrays of light generating sources.

FIG. 5 is a spectrum of the light emitted by another implementation of the device of FIG. 1, according to certain embodiments of the present disclosure, when measured at 5 cm from the light source.

FIG. 6 shows an exemplary block diagram of a computing device for performing any functions according to certain embodiments of the present disclosure.

FIG. 7A is an emission spectrum showing the power intensity over time of the light treatment applied to cells to assess angiogenesis (Example 1).

FIG. 7B is a blown up view of FIG. 7A.

FIG. 8 illustrates the decrease in power density over emission time and at different distances from the light source of a fluorescent light emitted by a photoactivated composition by a device according to certain embodiments of the present disclosure (Example 3).

FIG. 9 illustrates the increase in power density over emission time and at different distances from the light source of light transmitted through a photoactivatable composition by a device according to certain embodiments of the present disclosure (Example 3).

### DETAILED DESCRIPTION

To provide an understanding of the devices, lamps, and methods described herein, certain illustrative embodiments will now be described. For the purpose of clarity and illustration, the embodiments are described primarily with respect to light emitting diode (LED) light sources. However, it will be understood by one of ordinary skill in the art that other light sources may also be used in the devices, lamps, and methods described herein, which may be adapted and modified as appropriate. Such other additions and modifications will not depart from the scope hereof.

According to a first broad aspect of the present disclosure, there is provided a device 100 for emitting light, the device 100 is a lamp comprising: a first light source and a second light source. Specifically, the first light source, which in one embodiment is a set of first LEDs 102, has an emission spectra which can induce a therapeutic effect on a treatment area irradiated with the light source. The therapeutic effect can include an antimicrobial effect and/or a stimulatory effect such as initially increasing blood flow at the treatment site, followed by a reduction in inflammation and collagen production. The second light source, which in one embodiment is a set of second LEDs 104, has an emission spectra which can activate a photoactivatable composition applied on or located near the treatment area, or which may have an effect on local blood flow modulation or collagen remodeling.

The photoactivatable composition generally includes a photoactive agent which when activated can provide a therapeutic effect on the treatment area either by emission of fluorescent light at therapeutic wavelengths and at therapeutic intensities, by emission of energy which can then activate further photoactive agents in the compositions or the treatment site to have a therapeutic effect, and/or by excitation of its molecules from a singlet state to an excited singlet state which can then react with other molecules to produce for example reactive oxygen species. It is believed that low levels of reactive oxygen species can have a therapeutic effect on tissues. Examples of photoactivatable compositions are described in U.S. Patent Application Publication No. 2007/0128132, filed on Nov. 9, 2006, PCT Publication No. WO/2010/051636, filed on Nov. 6, 2009, PCT Publication No. WO/2010/051641, filed on Nov. 6, 2009, and PCT Application No. PCT/CA/2010/001134, filed on Jul. 19, 2010, the contents of which are herein incorporated by reference.

The first and second sets of LEDs 102, 104 can provide a complementary phototherapeutic treatment to the treatment site, whereby the therapeutic effect of the first set of LEDs 102 is augmented and complemented by the therapeutic effect of the second set of LEDs 104 through, for example, activation of a photoactivatable and therapeutic composition. In some embodiments, a fluorescence emitted by such a photoactivatable composition has a power density at the skin surface of less than about 75 mW/cm², less than about 50 mW/cm², less than about 10 mW/cm², less than about 5 mW/cm², less than about 2.5 mW/cm², or less than about 2 mW/cm². The maximum power density can be from about 0.02 mW/cm² to about 75 mW/cm², from about 0.02 mW/cm² to about 50 mW/cm², from about 0.02 mW/cm² to about 10 mW/cm², from about 0.02 mW/cm² to about 5 mW/cm², or from about 0.02 mW/cm² to about 10 mW/cm².

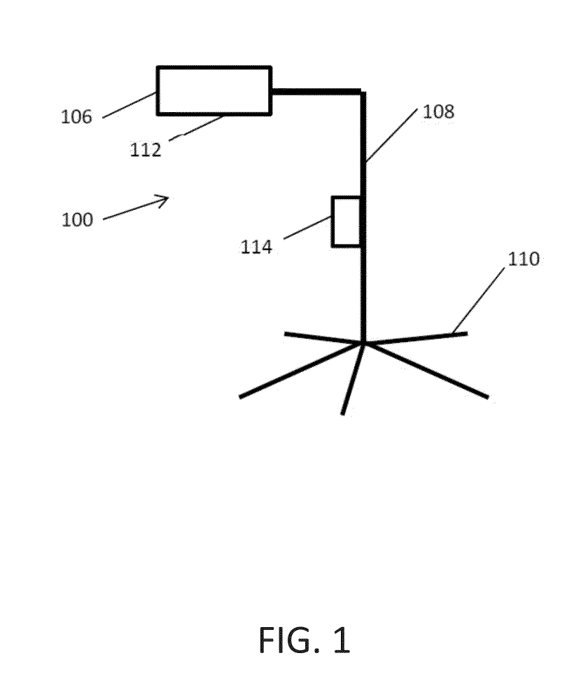

Referring now to an embodiment of the device 100 illustrated in FIG. 1, where the first and second set of LEDs 102, 104 are housed in a head 106 of the device which is adjustably attached to a body 108 having a base 110. The LEDs are mounted on a panel (not shown) within the housing as an array and emit light from an emitting surface 112 of the head 106. Optionally, depending on the power of the LEDs used and the treatment time, in order to minimize heating, the head 100 may include one or more heat sinks (not shown) and one or more fans (not shown) for cooling the first and/or the second set of LEDs. The device also includes a controller 114 in electronic communication with the LEDs for controlling various parameters of the emitted light such as power on/power off to individual LEDs within a set as well as to the different LED sets. Other parameters which may be controlled by the controller are: the maximum emitted power density of light from each LED, the bandwidth, the peak wavelength of emission, duration of light emission, variation of emitted light power density as a function of light emission time, variation of wavelength as a function of light emission time. This can allow a device operator to tailor the phototherapeutic treatment of each subject according to the therapy required.

The head 106 may be removable from the rest of the device 100 and replaceable. In certain embodiments, the lamp includes rotation device(s) (not shown) that allow for the head 106 to face any direction at any angle for convenience. The device 100 may include lockable wheels at the base (not shown) such that a user may freely move the device to a desired location and lock the device's position.

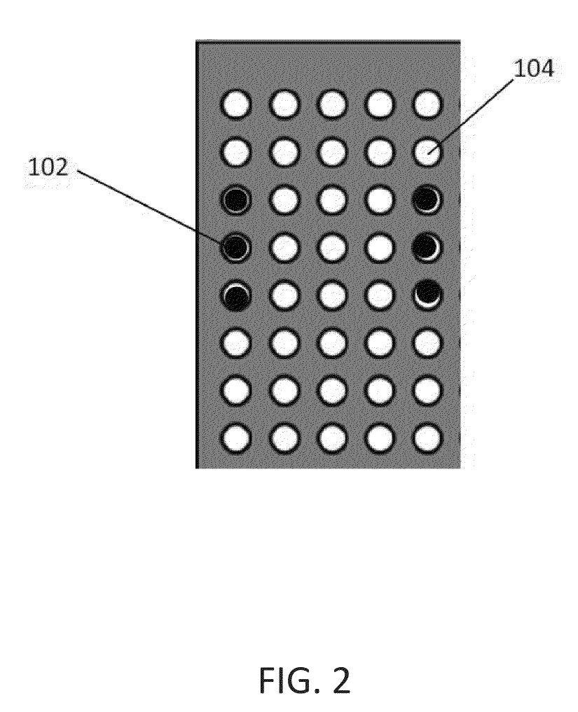

An exemplary physical spatial arrangement of the array of the first and second sets of LEDs 102, 104 are illustrated in FIG. 2. The array comprises 40 LEDs arranged in 8 rows and 5 columns with 6 LEDs in the first set and 34 LEDs in the second set. It will be understood by a skilled person that any other number of LEDs with any other array configuration is also possible. In another embodiment, the LED array comprises a total of at least 36 LEDs, at least 40 LEDS, at least 46 LEDs or at least 184 LEDs, mounted on the panel. In one embodiment, the LED array comprises 184 LEDs arranged as 8 rows and 23 columns. In yet another embodiment, the LED array comprises 36 LEDs comprising 6 rows by 6 columns. It will be appreciated by the skilled man that the LED array can be arranged in any configuration and can be any size or shape such as rectangular, circular or any other shape. The panel surface may be graduated so that some of the LEDs are closer to the treatment site than others. For example, may be a rectangular panel comprising the second set of LEDs on a first surface, and the first set of LEDs being mounted around the periphery of the first surface and spaced from the first surface.

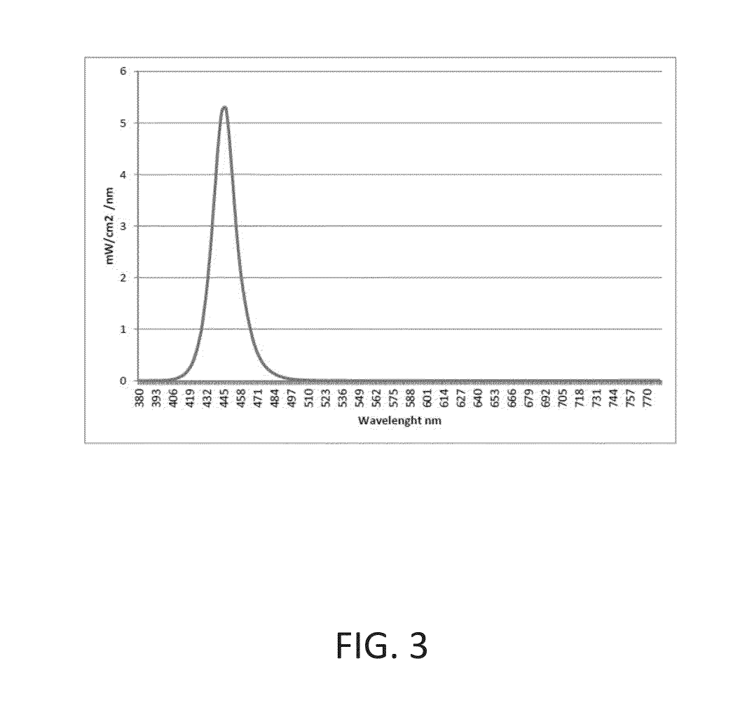

In the embodiment of FIG. 2, the first and second sets of LEDs have a different peak emitted wavelength from one another. The first set of LEDs has a peak emission wavelength of about 410 nm to about 420 nm and a bandwidth of about 13-15 nm, which may have an antibacterial effect against certain bacteria such as p. acnes, and the second set of LEDs has a peak emission wavelength of about 440 nm to about 470 nm and a bandwidth of about 20 nm±2 nm. FIG. 3 is an exemplary emission spectra of this embodiment of the device measured at 5 cm. Typically, the total emitted power density obtained at a distance of 5 cm with this embodiment of the device is about 60 to 150 mW/cm² with a total energy emitted over 5 minutes of about 40 to 50 J/cm².

In a different embodiment (not shown), the LEDs are mounted on a flexible substrate which may form part of, or be included with, a dressing, a mask, or any other material which can be applied to skin, hair, nails or other tissues.

The device head 106 may comprise a plurality of connectable panels (not shown) having LEDs mounted thereon. There may be any number of panels connectable together, such as 3, 4, 5, 6 or 7. The panels can be connected together such that their emitting surfaces 112 are angled with respect to one another. In this way, it may be possible to radiate light to different sides of a curved or irregular treatment site of a subject such as face, arms, legs. The size and shape of the panel will depend on the area to be treated. For example, for treatment of the face, a single curved panel, or a number of joined panels with a curvature can be used. The panels can be moveably connected to one another.

The LED array may also include a third set of LEDs that emits light at a different peak wavelength or power density or bandwidth than the first and/or second set of LEDs. In some embodiments, the third set of LEDs emits light in the red portion of the visible electromagnetic spectrum (e.g., between 630 nm and 700 nm). In other embodiments, the third set of LEDs emits light in the orange portion of the visible electromagnetic spectrum (e.g., between 635 nm and 590 nm). In yet other embodiments, the third set of LEDs emits light in a yellow portion of the visible electromagnetic spectrum (e.g., between 590 nm and 560 nm). In other embodiments, the third set of light generating sources emits light in the infrared portion of the electromagnetic spectrum (e.g., between 800 nm and 1000 nm). Alternative embodiments include LEDs that emit light of different wavelengths and power intensities than those described above, and which have different therapeutic effects.

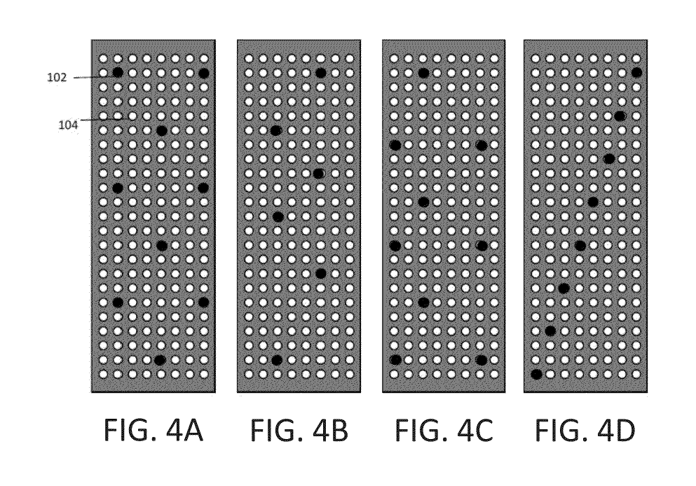

Further exemplary arrays of the first and second sets of LEDs 102, 104 are illustrated in FIG. 4. Different numbers and ratios of the number of LEDs in each of the first and second set of LEDs are possible. As the emitted power density of different LEDS can vary, the ratio can also be defined in terms of an emitted power density ratio of the first and second set of LEDs. The number or the power density ratio can be tailored according to the therapeutic effect desired using the device 100. For example, for an infected skin condition, the relative density of the first LED set can be increased, whereas for a cosmetic treatment the relative emitted power density of the second LED set may be increased.

Optionally, the device 100 may include a removable mask (cover) to reduce the size of the emitting area of the head 100. The device may be configured to deliver light with substantially equal distribution across an exposed surface with or without use of the mask. The head may have a U-shape, a circular shape, or any other suitable shape for a lamp head. The device 100 may also include filters to filter the light emitted from the emitting surface 112 in order to emit light of an appropriate wavelength, bandwidth or power density.

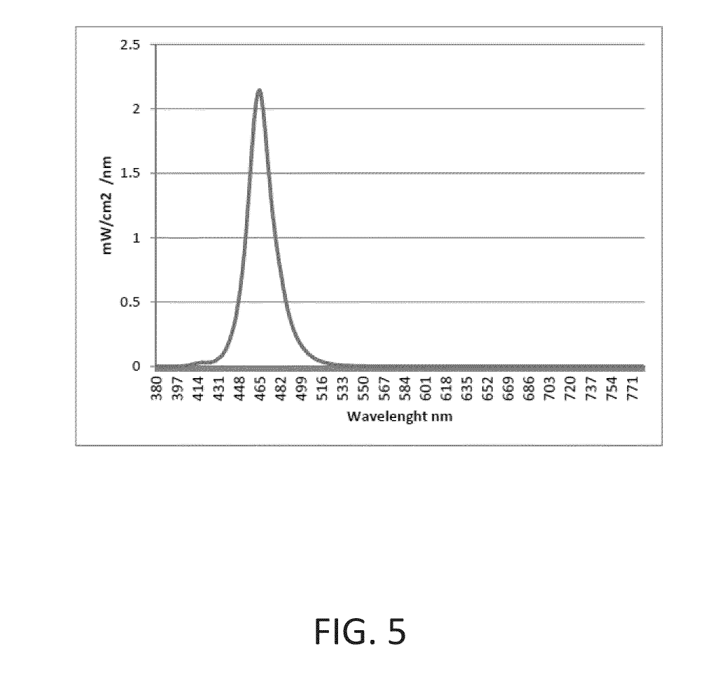

FIG. 5 is an emission spectra of a different embodiment of the device, which differs from the embodiment described above in that the second set of LEDs has a higher peak emission wavelength, of 450 to about 480 nm, and the head has a curved emission surface with the LED array mounted on a curved panel e.g. to follow the curved contour of a body part such as the face, arms, legs. In this embodiment, the average power density at a treatment distance is less than about 75 mW/cm², or about 30 to 150 mW/cm2.

In certain embodiments, the device may be configured to maintain a temperature of the exposed surface below some threshold, such as 40 degrees Celsius. For example, the light parameters may be adjusted when a determination is made that the emitted energy exceeds some threshold. In addition, the device may include a cooling device for eliminating heat in regions outside the desired area to be illuminated. For example, the cooling device may be a cooling mechanism for cooling the periphery of the desired area, or the cooling device may be a shield for absorbing the energy emitted by the device.

Referring now to the controller 114, by means of the controller, different treatment parameters can be pre-set as a treatment mode, or can be customized by the device operator. For example, the controller 114 may allow the user to select specific red, yellow, blue and/or infrared wavelengths, or a combination thereof to treat various conditions, such as skin conditions or wounds. Additional light color types may also be used. The controller 114 may optionally include a display (not shown) that assists a user in selecting and controlling treatment modes, timers, and other functionality features.

The controller 114 can illuminate different LEDs at different wavelength ranges simultaneously or at separate times. It may be desirable to activate two wavelength ranges simultaneously such that both effects take place simultaneously. In addition, the combination of the two wavelength ranges may introduce a synergistic effect such that simultaneous application of multiple light generating sources operating in different wavelength ranges may result in more efficient treatment than the single application of either wavelength range at a time. Alternatively, it may be desirable to activate one wavelength range at a time. Operating a phototherapeutic lamp within a single wavelength range may be desirable if no synergistic effects are expected from operating at multiple wavelength ranges simultaneously, or if it is determined that doing so has a detrimental effect. It may also be desirable to alternate between two or more wavelength ranges. For example, in weekly treatments for acne, alternating between two wavelength ranges (e.g., 633 nm (red) and 415 nm (violet)) may result in more efficient treatment than using one wavelength range alone.

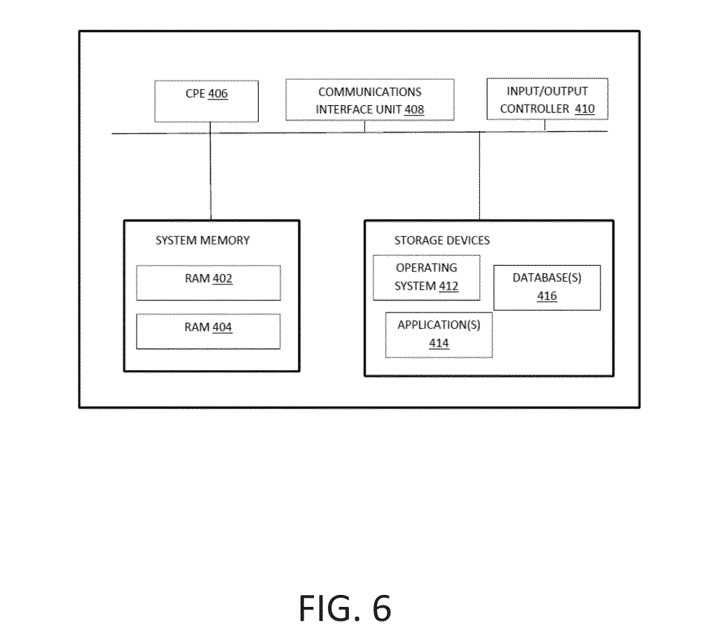

Treatment modes may be stored on a memory device or database in a machine readable form as described in relation to the device in FIG. 6. For example, a user may select one or more of a list of skin conditions to be treated. With treatment modes stored on a memory device or database, the lamp controller can access operating parameters of the phototherapeutic lamp that correspond with a particular light therapy treatment. Such parameters may be inputted by a manufacturer or programmer of the device, or alternatively a user may provide adjustment operating parameter in accordance with a customized phototherapeutic skin treatment program.

The circuitry in the device may include a switch to select a mode of operation. The switch may be implemented in hardware, software, firmware, or a combination thereof. Different modes of operation may specify various parameters of the generated light, such as the wavelength range, bandwidth, peak wavelength, or any other light source parameter. In addition, a light source may be configured to produce pulses of light. In pulsating light sources, stronger intensities may be used to deliver a same amount of energy in a same amount of time as a nonpulsating light source. Pulsating may therefore be desirable for delivering stronger intensity light for a short amount of time and may accelerate the efficiency of a treatment. In this case, different modes may include different pulsation parameters, such as the pulse duration, the pulse frequency, the pulse intensity, the number of pulses, or any other pulsation parameter. In some cases, it may be desirable to keep the pulse parameters constant throughout a treatment session, such that the same pulse is delivered repeatedly. In other cases, it may be desirable to vary one or more pulse parameters over time across multiple pulses within the same session. For example, it may be desirable to operate in a mode where the pulse intensity increases with each pulse. In another example, it may be desirable to alternate between two or more wavelength ranges with each pulse. The parameters of the generated light (non-pulsating or pulsating) may be adjusted for any number of paradigms.

Different modes of operation may also include illuminating different LEDs at different times. For example, only one of the LED sets may be illuminated in a mode, or a selected number of the LEDs within a set. This may be desired if the area requiring treatment (e.g., a wound) is small such that illuminating all the LEDs would treat a larger region than required. As an example, it may be desirable to treat an area of skin near the eye, but delivering light to the eye may cause damage. In this case, using a subset of light generating sources is useful to appropriately control the size and shape of the region to be treated. In another example, when a mode includes using pulses, two subsets of the light generating sources may alternately pulse on and off such that only one subset of sources is illuminated at any given time. This mode may be selected when it is desirable to deliver a transient light with strong intensity to a subset of locations.

The device operator may directly select a mode of operation, or the device may include a user interface that allows the user to select one or more goals, and the device may be configured to select an appropriate mode based on the user's selection. For example, the device operator may indicate at the user interface that it is desirable to use a mode for treating acne, and an appropriate mode may be selected, such as a non-pulsating emission with a first emitted maximum peak of about 400 nm to about 430 nm at a full width half maximum bandwidth of about 14 nm, and a second maximum peak of about 440 to about 470 nm, or any other suitable mode for treating acne.

The modes selected by the circuitry of the device or by the operator may be appropriately adjusted to be within levels that are safe and comply with regulatory requirements in any country.

FIG. 6 is a block diagram of a computing device, which may be included in the device, for performing any of the processes described herein. Each of the components of these systems may be implemented on one or more computing devices 400. In certain aspects, a plurality of the components of these systems may be included within one computing device 400. In certain implementations, a component and a storage device may be implemented across several computing devices 400.

The computing device 400 comprises at least one communications interface unit, an input/output controller 410, system memory, and one or more data storage devices. The system memory includes at least one random access memory (RAM 402) and at least one read-only memory (ROM 404). All of these elements are in communication with a central processing unit (CPU 406) to facilitate the operation of the computing device 400. The computing device 400 may be configured in many different ways. For example, the computing device 400 may be a conventional standalone computer or alternatively, the functions of computing device 400 may be distributed across multiple computer systems and architectures. In FIG. 4, the computing device 400 is linked, via network or local network, to other servers or systems.

The computing device 400 may be configured in a distributed architecture, wherein databases and processors are housed in separate units or locations. Some units perform primary processing functions and contain at a minimum a general controller or a processor and a system memory. In distributed architecture implementations, each of these units may be attached via the communications interface unit 408 to a communications hub or port (not shown) that serves as a primary communication link with other servers, client or user computers and other related devices. The communications hub or port may have minimal processing capability itself, serving primarily as a communications router. A variety of communications protocols may be part of the system, including, but not limited to: Ethernet, SAP, SAS™, ATP, BLUETOOTH™, GSM and TCP/IP.

The CPU 406 comprises a processor, such as one or more conventional microprocessors and one or more supplementary co-processors such as math co-processors for offloading workload from the CPU 406. The CPU 406 is in communication with the communications interface unit 408 and the input/output controller 410, through which the CPU 406 communicates with other devices such as other servers, user terminals, or devices. The communications interface unit 408 and the input/output controller 410 may include multiple communication channels for simultaneous communication with, for example, other processors, servers or client terminals.

The CPU 406 is also in communication with the data storage device. The data storage device may comprise an appropriate combination of magnetic, optical or semiconductor memory, and may include, for example, RAM 402, ROM 404, flash drive, an optical disc such as a compact disc or a hard disk or drive. The CPU 406 and the data storage device each may be, for example, located entirely within a single computer or other computing device; or connected to each other by a communication medium, such as a USB port, serial port cable, a coaxial cable, an Ethernet cable, a telephone line, a radio frequency transceiver or other similar wireless or wired medium or combination of the foregoing. For example, the CPU 406 may be connected to the data storage device via the communications interface unit 408. The CPU 406 may be configured to perform one or more particular processing functions.

The data storage device may store, for example, (i) an operating system 412 for the computing device 400; (ii) one or more applications 414 (e.g., computer program code or a computer program product) adapted to direct the CPU 406 in accordance with the systems and methods described here, and particularly in accordance with the processes described in detail with regard to the CPU 406; or (iii) database(s) 416 adapted to store information that may be utilized to store information required by the program.

The operating system 412 and applications 414 may be stored, for example, in a compressed, an uncompiled and an encrypted format, and may include computer program code. The instructions of the program may be read into a main memory of the processor from a computer-readable medium other than the data storage device, such as from the ROM 404 or from the RAM 402. While execution of sequences of instructions in the program causes the CPU 406 to perform the process steps described herein, hard-wired circuitry may be used in place of, or in combination with, software instructions for implementation of the processes of the present disclosure. Thus, the systems and methods described are not limited to any specific combination of hardware and software.

Suitable computer program code may be provided for performing one or more functions in relation to selecting a mode of operation as described herein. The program also may include program elements such as an operating system 412, a database management system and “device drivers” that allow the processor to interface with computer peripheral devices (e.g., a video display, a keyboard, a computer mouse, etc.) via the input/output controller 410.

The term “computer-readable medium” as used herein refers to any non-transitory medium that provides or participates in providing instructions to the processor of the computing device 400 (or any other processor of a device described herein) for execution. Such a medium may take many forms, including but not limited to, non-volatile media and volatile media. Non-volatile media include, for example, optical, magnetic, or opto-magnetic disks, or integrated circuit memory, such as flash memory. Volatile media include dynamic random access memory (DRAM), which typically constitutes the main memory. Common forms of computer-readable media include, for example, a floppy disk, a flexible disk, hard disk, magnetic tape, any other magnetic medium, a CD-ROM, DVD, any other optical medium, punch cards, paper tape, any other physical medium with patterns of holes, a RAM, a PROM, an EPROM or EEPROM (electronically erasable programmable read-only memory), a FLASH-EEPROM, any other memory chip or cartridge, or any other non-transitory medium from which a computer can read.

Various forms of computer readable media may be involved in carrying one or more sequences of one or more instructions to the CPU 406 (or any other processor of a device described herein) for execution. For example, the instructions may initially be borne on a magnetic disk of a remote computer (not shown). The remote computer can load the instructions into its dynamic memory and send the instructions over an Ethernet connection, cable line, or even telephone line using a modem. A communications device local to a computing device 400 (e.g., a server) can receive the data on the respective communications line and place the data on a system bus for the processor. The system bus carries the data to main memory, from which the processor retrieves and executes the instructions. The instructions received by main memory may optionally be stored in memory either before or after execution by the processor. In addition, instructions may be received via a communication port as electrical, electromagnetic or optical signals, which are exemplary forms of wireless communications or data streams that carry various types of information.

### Methods

Methods for treating a subject's skin, wound, lesion or other skin condition are also disclosed. The therapeutic benefits of the light reaching the subject's skin can be related to the wavelength of light and power density of the emitted light as well as the total emitted power over the treatment time.

In certain aspects, the method includes irradiating the subject's skin with light having a power density of about 10 mW/cm² to about 150 mW/cm². In certain embodiments, when applying two or more sets of LEDs with different wavelength ranges, the power density of one set of LEDs may be restricted to be less than a threshold amount of another set of LEDs. For example, the power density of one set may be restricted to be less than 10% of the power density of another set, or any other suitable threshold amount.

In certain embodiments, light is applied to a treatment area for a period of 1 second to 30 minutes. In certain embodiments, light is applied for a period of 1-30 seconds, 15-45 seconds, 30-60 seconds, 0.75-1.5 minutes, 1-2 minutes, 1.5-2.5 minutes, 2-3 minutes, 2.5-3.5 minutes, 3-4 minutes, 3.5-4.5 minutes, 4-5 minutes, 4-6 minutes, 5-7 minutes, 6-8 minutes, 7-9 minutes, 8-10 minutes, 9-11 minutes, 10-12 minutes, 11-13 minutes, 12-14 minutes, 13-15 minutes, 14-16 minutes, 15-17 minutes, 16-18 minutes, 17-19 minutes, 18-20 minutes, 19-21 minutes, 20-22 minutes, 21-23 minutes, 22-24 minutes, 23-25 minutes, 24-26 minutes, 25-27 minutes, 26-28 minutes, 27-29 minutes, or 28-30 minutes. The treatment period will depend on the total joules of light energy delivered to the treatment site, so a higher emitted light power density will require a shorter time, and vice versa.

In some embodiments, the method for treating a subject's skin, wound, lesion or other skin condition further includes applying a photoactivatable composition to a subject's skin prior to applying light of a certain wavelength and power density, for example from an embodiment of the present device 100. The photoactivatable composition may include one or more compositions. For example, the photoactivatable composition may include a a photoactivator component which can be activated by light of specific wavelength (i.e., actinic light). The photoactivator component comprises one or more photoactivator molecules which are activated by actinic light and accelerate the dispersion of light energy, which leads to the photoactivator carrying on a therapeutic effect on its own, or to the photochemical activation of other agents contained in the composition that could carry on a therapeutic effect (e.g., acceleration in the breakdown process of an oxidant such as peroxide) when such compound is present in the composition). The included photoactivators are illuminated by photons of a certain wavelength and excited to a higher energy state. When the photoactivators' excited electrons return to a lower energy state, they emit photons with a lower energy level, thus causing the emission of light of a longer wavelength (Stokes shift). In the proper environment, much of this energy transfer is transferred to the other components of the photoactivatable composition or to the treatment site directly.

Suitable photoactivators can be fluorescent dyes (or stains), although other dye groups or dyes (biological and histological dyes, food colorings, carotenoids) can also be used. Combining photoactivators may increase photo-absorbtion by the combined dye molecules and enhance absorption and photo-biomodulation selectivity. Combining photoactivators may also result in a transfer of energy between the photoactivators. This creates multiple possibilities of generating new photosensitive, and/or selective photoactivator mixtures. Suitable photoactivators may include:

### Chlorophyll Dyes

Exemplary chlorophyll dyes include but are not limited to chlorophyll a; chlorophyll b; oil soluble chlorophyll; bacteriochlorophyll a; bacteriochlorophyll b; bacteriochlorophyll c; bacteriochlorophyll d; protochlorophyll; protochlorophyll a; amphiphilic chlorophyll derivative 1; and amphiphilic chlorophyll derivative 2.

### Xanthene Derivatives

Exemplary xanthene dyes include but are not limited to Eosin B (4′,5′-dibromo,2′,7′-dinitr-o-fluorescein, dianion); eosin Y; eosin Y (2′,4′,5′,7′-tetrabromo-fluoresc-ein, dianion); eosin (2′,4′,5′,7′-tetrabromo-fluorescein, dianion); eosin (2′,4′,5′,7′-tetrabromo-fluorescein, dianion) methyl ester; eosin (2′,4′,5′,7′-tetrabromo-fluorescein, monoanion) p-isopropylbenzyl ester; eosin derivative (2′,7′-dibromo-fluorescein, dianion); eosin derivative (4′,5′-dibromo-fluorescein, dianion); eosin derivative (2′,7′-dichloro-fluorescein, dianion); eosin derivative (4′,5′-dichloro-fluorescein, dianion); eosin derivative (2′,7′-diiodo-fluorescein, dianion); eosin derivative (4′,5′-diiodo-fluorescein, dianion); eosin derivative (tribromo-fluorescein, dianion); eosin derivative (2′,4′,5′,7′-tetrachlor-o-fluorescein, dianion); eosin; eosin dicetylpyridinium chloride ion pair; erythrosin B (2′,4′,5′,7′-tetraiodo-fluorescein, dianion); erythrosin; erythrosin dianion; erythiosin B; fluorescein; fluorescein dianion; phloxin B (2′,4′,5′,7′-tetrabromo-3,4,5,6-tetrachloro-fluorescein, dianion); phloxin B (tetrachloro-tetrabromo-fluorescein); phloxine B; rose bengal (3,4,5,6-tetrachloro-2′,4′,5′,7′-tetraiodofluorescein, dianion); pyronin G, pyronin J, pyronin Y; Rhodamine dyes such as rhodamines include 4,5-dibromo-rhodamine methyl ester; 4,5-dibromo-rhodamine n-butyl ester; rhodamine 101 methyl ester; rhodamine 123; rhodamine 6G; rhodamine 6G hexyl ester; tetrabromo-rhodamine 123; and tetramethyl-rhodamine ethyl ester.

### Methylene Blue Dyes

Exemplary methylene blue derivatives include but are not limited to 1-methyl methylene blue; 1,9-dimethyl methylene blue; methylene blue; methylene blue (16 .mu.M); methylene blue (14 .mu.M); methylene violet; bromomethylene violet; 4-iodomethylene violet; 1,9-dimethyl-3-dimethyl-amino-7-diethyl-a-mino-phenothiazine; and 1,9-dimethyl-3-diethylamino-7-dibutyl-amino-phenot-hiazine.

### Azo Dyes

Exemplary azo (or diazo-) dyes include but are not limited to methyl violet, neutral red, para red (pigment red 1), amaranth (Azorubine S), Carmoisine (azorubine, food red 3, acid red 14), allura red AC (FD&C 40), tartrazine (FD&C Yellow 5), orange G (acid orange 10), Ponceau 4R (food red 7), methyl red (acid red 2), and murexide-ammonium purpurate.

In some aspects of the disclosure, the one or more photoactivator can be independently selected from any of Acid black 1, Acid blue 22, Acid blue 93, Acid fuchsin, Acid green, Acid green 1, Acid green 5, Acid magenta, Acid orange 10, Acid red 26, Acid red 29, Acid red 44, Acid red 51, Acid red 66, Acid red 87, Acid red 91, Acid red 92, Acid red 94, Acid red 101, Acid red 103, Acid roseine, Acid rubin, Acid violet 19, Acid yellow 1, Acid yellow 9, Acid yellow 23, Acid yellow 24, Acid yellow 36, Acid yellow 73, Acid yellow S, Acridine orange, Acriflavine, Alcian blue, Alcian yellow, Alcohol soluble eosin, Alizarin, Alizarin blue 2RC, Alizarin carmine, Alizarin cyanin BBS, Alizarol cyanin R, Alizarin red S, Alizarin purpurin, Aluminon, Amido black 10B, Amidoschwarz, Aniline blue WS, Anthracene blue SWR, Auramine O, Azocannine B, Azocarmine G, Azoic diazo 5, Azoic diazo 48, Azure A, Azure B, Azure C, Basic blue 8, Basic blue 9, Basic blue 12, Basic blue 15, Basic blue 17, Basic blue 20, Basic blue 26, Basic brown 1, Basic fuchsin, Basic green 4, Basic orange 14, Basic red 2, Basic red 5, Basic red 9, Basic violet 2, Basic violet 3, Basic violet 4, Basic violet 10, Basic violet 14, Basic yellow 1, Basic yellow 2, Biebrich scarlet, Bismarck brown Y, Brilliant crystal scarlet 6R, Calcium red, Carmine, Carminic acid, Celestine blue B, China blue, Cochineal, Coelestine blue, Chrome violet CG, Chromotrope 2R, Chromoxane cyanin R, Congo corinth, Congo red, Cotton blue, Cotton red, Croceine scarlet, Crocin, Crystal ponceau 6R, Crystal violet, Dahlia, Diamond green B, Direct blue 14, Direct blue 58, Direct red, Direct red 10, Direct red 28, Direct red 80, Direct yellow 7, Eosin B, Eosin Bluish, Eosin, Eosin Y, Eosin yellowish, Eosinol, Erie garnet B, Eriochrome cyanin R, Erythrosin B, Ethyl eosin, Ethyl green, Ethyl violet, Evans blue, Fast blue B, Fast green FCF, Fast red B, Fast yellow, Fluorescein, Food green 3, Gallein, Gallamine blue, Gallocyanin, Gentian violet, Haematein, Haematine, Haematoxylin, Helio fast rubin BBL, Helvetia blue, Hematein, Hematine, Hematoxylin, Hoffman's violet, Imperial red, Indocyanin green, Ingrain blue, Ingrain blue 1, Ingrain yellow 1, INT, Kermes, Kermesic acid, Kernechtrot, Lac, Laccaic acid, Lauth's violet, Light green, Lissamine green SF, Luxol fast blue, Magenta 0, Magenta I, Magenta II, Magenta III, Malachite green, Manchester brown, Martius yellow, Merbromin, Mercurochrome, Metanil yellow, Methylene azure A, Methylene azure B, Methylene azure C, Methylene blue, Methyl blue, Methyl green, Methyl violet, Methyl violet 2B, Methyl violet 10B, Mordant blue 3, Mordant blue 10, Mordant blue 14, Mordant blue 23, Mordant blue 32, Mordant blue 45, Mordant red 3, Mordant red 11, Mordant violet 25, Mordant violet 39 Naphthol blue black, Naphthol green B, Naphthol yellow S, Natural black 1, Natural red, Natural red 3, Natural red 4, Natural red 8, Natural red 16, Natural red 25, Natural red 28, Natural yellow 6, NBT, Neutral red, New fuchsin, Niagara blue 3B, Night blue, Nile blue, Nile blue A, Nile blue oxazone, Nile blue sulphate, Nile red, Nitro BT, Nitro blue tetrazolium, Nuclear fast red, Oil red O, Orange G, Orcein, Pararosanilin, Phloxine B, Picric acid, Ponceau 2R, Ponceau 6R, Ponceau B, Ponceau de Xylidine, Ponceau S, Primula, Purpurin, phycobilins, Phycocyanins, Phycoerythrins. Phycoerythrincyanin (PEC), Phthalocyanines, Pyronin B, Pyronin G, Pyronin Y, Rhodamine B, Rosanilin, Rose bengal, Saffron, Safranin O, Scarlet R, Scarlet red, Scharlach R, Shellac, Sirius red F3B, Solochrome cyanin R, Soluble blue, Solvent black 3, Solvent blue 38, Solvent red 23, Solvent red 24, Solvent red 27, Solvent red 45, Solvent yellow 94, Spirit soluble eosin, Sudan III, Sudan IV, Sudan black B, Sulfur yellow S, Swiss blue, Tartrazine, Thioflavine S, Thioflavine T, Thionin, Toluidine blue, Toluyline red, Tropaeolin G, Trypaflavine, Trypan blue, Uranin, Victoria blue 4R, Victoria blue B, Victoria green B, Water blue I, Water soluble eosin, Xylidine ponceau, or Yellowish eosin.

Photoactivatable compositions may contain other compounds, such as oxygen-rich agents, pH controlling agents (e.g., sodium acetate, sodium hydroxide), light diffracting agents (e.g., porcelain crystals, hydroxylapatite), healing factors (e.g., hyaluronic acid, glucosamine), chelating agents (e.g., EDTA, EGTA), lipolysis stimulating agents (e.g., caffeine), and/or hydrophilic gelling agents (e.g., glucose, celluloses).

When used in combination with a photoactivatable composition, it may be particularly useful for the array of LEDs to include more than one type of LED, each LED emitting at a different wavelength. For example, each LED may emit light at a wavelength that overlaps or matches the absorption band of the one or more chromophores in the photoactivatable composition. Each type of LED may be switched on and off independently.

In certain embodiments, the method includes i) applying a photoactivatable composition to a subject's skin, ii) applying light having a wavelength that overlaps an absorption spectra of the applied photoactivatable composition, wherein the light is applied for a period of time until the photoactivable composition is substantially photobleached.

In certain embodiments, the method includes i) applying a photoactivatable composition to a subject's skin, ii) applying a first light having a wavelength that overlaps an absorption spectra of the applied photoactivatable composition, wherein the first light is applied for a period of time until the photoactivable composition is substantially photo-bleached, and iii) applying a second light having a wavelength that is different than the first light.

In a particular embodiment, the method includes i) applying a photoactivatable composition to a subject's skin, wherein the photoactivatable composition absorbs light in the blue portion of the visible electromagnetic spectrum, ii) applying blue light to the subject's skin, wherein the blue light is applied until the photoactivatable composition is substantially photo-bleached, and iii) applying red light to the subject's skin.

In certain embodiments, the method comprises irradiating the tissue with a first light having a peak emission wavelength of about 400 to about 750 nm, and modulating at least one of the peak emission wavelength, bandwidth, power density or fluence of the first light during the irradiation of the tissue. In one implementation, the method comprises decreasing or increasing the maximum power intensity of the light emitted from at least one light source during the time of light irradiation. Lights from different light sources may be modulated differently, at different times or at the same time. It will be understood that that the modulation of light from one light source may occur over only a portion of the total irradiation time, or over the full irradiation time. In certain embodiments, the power density may be increased or decreased at a rate, for example, of at about 0.002 mW/cm² per minute of irradiation to about 0.1 mW/cm² per minute of irradiation, about 0.005 mW/cm² per minute, about 0.006 mW/cm² per minute, or about 0.012 mW/cm² per minute.

The method may comprise irradiating the tissue with a first light having a peak emission wavelength of about 400 to about 750 nm, and a power density of about 10 to about 75 mW/cm², or about 55 mW/cm² to about 150 mW/cm².

The method may comprise irradiating the tissue with a first light having a peak emission wavelength of about 400 to about 750 nm, and a bandwidth of about 19 nm±about 5 nm.

The method may comprise irradiating the tissue with a first light having a peak emission wavelength of about 400 to about 750 nm, and a fluence during a single treatment of about 4 to about 60 J/cm², about 10 to about 60 J/cm², about 10 to about 50 J/cm², about 10 to about 40 J/cm², about 10 to about 30 J/cm², about 20 to about 40 J/cm², or about 10 to about 20 J/cm². The treatment area may be irradiated simultaneously or at different times, from a single light source or a plurality of light sources with light having different properties. The irradiating light may have any of the properties described above in relation to aspects of the device and lamp.

The treatment time may range from about 30 seconds to about 30 minutes, typically 5 to 15 minutes. The maximum light intensity can be about 12 J/cm² per minute of treatment. The light may be applied continuously or pulsed.

In certain embodiments, the irradiating light is a fluorescence or phosphorescence light within one or more of the green, yellow, orange, red and infrared portions of the electromagnetic spectrum, for example having a peak wavelength within the range of about 490 nm to about 720 nm. In one embodiment, the irradiating light has a wavelength of between about 400 nm to about 700 nm, about 480 nm to about 700 nm, about 500 nm to about 660 nm, about 540 nm to about 640 nm. In another embodiment, the irradiating light has a power density of between 0.005 to about 10 mW/cm², about 0.5 to about 5 mW/cm². In certain embodiments, the irradiating light has a bandwidth of about 15 nm to about 100 nm, about 25 nm to about 80 nm, about 30 nm to about 70 nm, or about 20 nm to about 50 nm. The light source of the irradiating light may be a photoactive agent such as a fluorochrome which is activated by the first light source, or any other light source, to emit fluorescence. Alternatively, the irradiating light may be from an electronically generated light such as LED, laser etc which mimics a fluorescence or phosphorescence spectra.

In certain embodiments, the maximum power density of the irradiating light is from about 0.01 mW/cm² to about 200 mW/cm², 0.02 mW/cm² to about 150 mW/cm², 0.02 mW/cm² to about 135 mW/cm², 0.02 mW/cm² to about 75 mW/cm², 0.02 mW/cm² to about 60 mW/cm², about 0.02 mW/cm² to about 50 mW/cm², about 0.02 mW/cm² to about 30 mW/cm², about 0.02 mW/cm² to about 15 mW/cm².

In certain other embodiments, the light generated from the array of LEDs or other light source is pulsed. In certain embodiments, the light generated has a pulse duration between about 10 ms and about 300 ms. However, a longer and shorter pulse duration can be used depending on the application. In some embodiments, the light generated has a pulse duration between about 20 ms and about 100 ms. In some embodiments, the light generated has a pulse duration between about 20 ms and about 60 ms. In some embodiments, the beam of radiation has a pulse duration between about 20 ms and about 40 ms. In some embodiments, the light generated has a pulse duration between about 40 ms and about 60 ms. In some embodiments, the light generated has a pulse duration of about 40 ms. In some embodiments, the light generated has a pulse duration greater than about 40 ms. In some embodiments, the light generated has a pulse duration of less than 1 ms, and preferably less than 500 ns. In addition, the pulse duration may be dependent on other characteristics of the generated light, such as the amplitude, wavelength, bandwidth, or a combination thereof.

The method may also include filtering, attenuating, amplifying, polarizing, or otherwise modifying the emitted light by one or more optical elements before it reaches an area of tissue, e.g. skin, to which it is directed. For example, the light may be filtered by a filter which removes a certain bandwidth of light such as a UV filter.

In certain embodiments, the phototherapeutic device may also be used in combination with a shield that effectively blocks the light being emitted from the LEDs of the device. For example, when only a portion of the subject's skin surface requires treatment, a shield may be used to prevent the emitted light from being applied to the area of skin not requiring treatment.

### Uses

The systems, devices, and methods of the present disclosure enable the use of light therapy technology for a variety of cosmetic, health and medical applications. Photomodulation of cellular activity induced by light has been found beneficial in skin therapy or treatment methods. The systems, devices and methods of the present disclosure may be useful in the treatment of a wound and tissue repair, skin condition, skin rejuvenation and skin maintenance and acute inflammation.

“Skin rejuvenation” means a process of reducing, diminishing, retarding or reversing one or more signs of skin aging. For instance, common signs of skin aging include, but are not limited to, appearance of fine lines or wrinkles, thin and transparent skin, loss of underlying fat (leading to hollowed cheeks and eye sockets as well as noticeable loss of firmness on the hands and neck), bone loss (such that bones shrink away from the skin due to bone loss, which causes sagging skin), dry skin (which might itch), inability to sweat sufficiently to cool the skin, unwanted facial hair, freckles, age spots, spider veins, rough and leathery skin, fine wrinkles that disappear when stretched, loose skin, or a blotchy complexion. According to the present disclosure, one or more of the above signs of aging may be reduced, diminished, retarded or even reversed by the devices, methods, uses and systems of the present disclosure.

“Skin disorders” include, but are not limited to, erythema, telangiectasia, actinic telangiectasia, psoriasis, skin cancer, pemphigus, sunburn, dermatitis, actinic keratosis, eczema, rashes, acne, impetigo, lichen simplex chronicus, rhinophyma, perioral dermatitis, diffuse sebaceous glands hyperplasia, other sebaceous gland disorders, collagen-related skin diseases (connective tissue disorders), other sweat gland disorders, granulomatous skin conditions, vascular lesions, benign pigmented lesions, hair disorders and some skin infections, chronic and acute inflammation, pseudofolliculitis barbae, drug eruptions, erythema multiforme, erythema nodosum, granuloma annulare, actinic keratosis, purpura, alopecia areata, aphthous stomatitis, drug eruptions, dry skin, neoplastic disorders, chapping, xerosis, ichthyosis vulgaris, fungal infections, herpes simplex, intertrigo, keloids, keratoses, milia, moluscum contagiosum, pityriasis rosea, pruritus, urticaria, and vascular tumors and malformations. Dermatitis includes contact dermatitis, atopic dermatitis, seborrheic dermatitis, nummular dermatitis, generalized exfoliative dermatitis, and statis dermatitis. Skin cancers include melanoma, basal cell carcinoma, and squamous cell carcinoma.

Some types of acne include, for example, acne vulgaris, cystic acne, acne atrophica, bromide acne, chlorine acne, acne conglobata, acne cosmetica, acne detergicans, epidemic acne, acne estivalis, acne fulminans, halogen acne, acne indurata, iodide acne, acne keloid, acne mechanica, acne papulosa, pomade acne, premenstral acne, acne pustulosa, acne scorbutica, acne scrofulosorum, acne urticata, acne varioliformis, acne venenata, propionic acne, acne excoriee, gram negative acne, steroid acne, and nodulocystic acne.

Some skin disorders present various symptoms including redness, flushing, burning, scaling, pimples, papules, pustules, comedones, macules, nodules, vesicles, blisters, telangiectasia, spider veins, sores, surface irritations or pain, itching, inflammation, red, purple, or blue patches or discolorations, moles, and/or tumors.

“Wound” means an injury to any tissue, including for example, acute, subacute, delayed or difficult to heal wounds, and chronic wounds. Examples of wounds may include both open and closed wounds. Wounds include, for example, burns, incisions, excisions, lacerations, abrasions, puncture or penetrating wounds, surgical wounds, contusions, hematomas, crushing injuries, sores (such as for example pressure sores), ulcers, wounds caused by periodontitis (inflammation of the periodontium). Ulcers can include diabetic foot ulcers, pressure ulcers, amputations, venous ulcers, chronic ulcers and/or any wound that may be classified as being Grade I through Grade IV wounds.

“Acute inflammation” can present itself as pain, heat, redness, swelling and loss of function. It includes those seen in allergic reactions such as insect bites e.g.; mosquito, bees, wasps, poison ivy, post-ablative treatment.

Identification of equivalent devices, methods and uses are well within the skill of the ordinary practitioner and would require no more than routine experimentation, in light of the teachings of the present disclosure. Practice of the disclosure will be still more fully understood from the following examples, which are presented herein for illustration only and should not be construed as limiting the disclosure in any way.

### EXAMPLES

The examples below are given so as to illustrate the practice of various embodiments of the present disclosure. They are not intended to limit or define the entire scope of this disclosure.

### Example 1

### Angiogenic Potential of Light Treatment Using Embodiments of the Present Disclosure

A human skin model was developed to assess the angiogenic potential of the biophotonic material of the present disclosure. Briefly, a biophotonic composition comprising Eosin was placed on top of a human skin model containing fibroblasts and keratinocytes. The skin model and the composition were separated by a nylon mesh of 20 micron pore size. The composition was then irradiated with blue light (activating light′) for 5 minutes at a distance of 5 cm from the light source. The activating light consisted of light emitted from an LED lamp having an average peak wavelength of about 400-470 nm, and a power intensity measured at 10 cm of 7.7 J/cm² to 11.5 J/cm². Upon illumination with the activating light, the biophotonic composition emitted fluorescent light, which may be replicated by a device, system or use of the present disclosure. Since the biophotonic composition was in limited contact with the cells, the fibroblasts and keratinocytes were exposed mainly to the activating light and the fluorescent light emitted from the biophotonic composition. Conditioned media from the treated human 3D skin model were then applied to human aortic endothelial cells previously plated in matrigel. The formation of tubes by endothelial cells was observed and monitored by microscopy after 24 hours. The conditioned medium from 3D skin models treated with light illumination induced endothelial tube formation in vitro, suggesting an indirect effect of the light treatment (blue light and fluorescence) on angiogenesis via the production of factors by fibroblasts and keratinocytes. Plain medium and conditioned medium from untreated skin samples were used as a control, and did not induce endothelial tube formation.

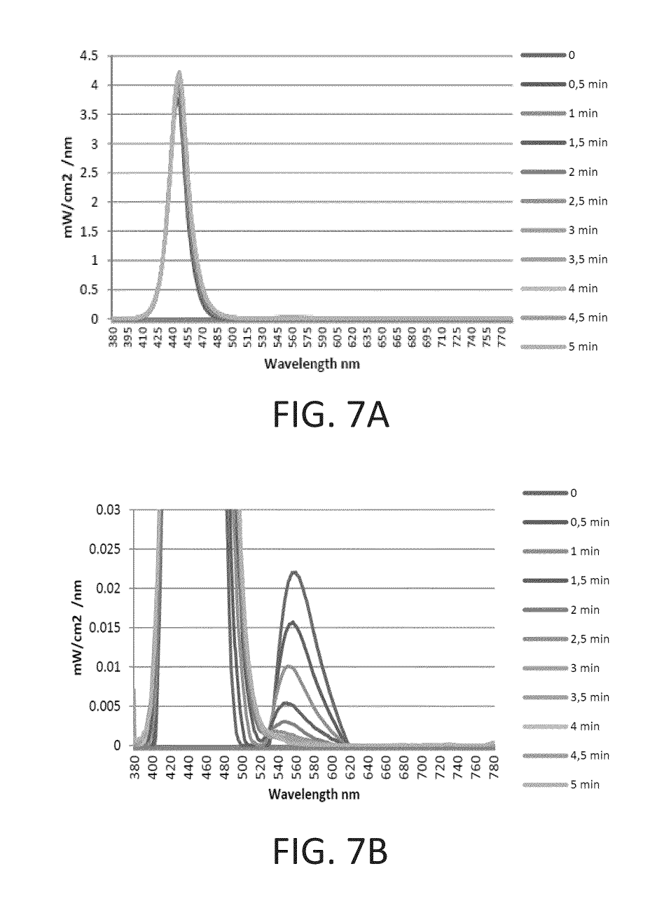

FIGS. 7a and 7b are emission spectra showing the power intensity over time of the light treatment applied to the cells in this Example. The emitted fluorescent light was about 0.3% of the total light intensity (about 30-35 J/cm²) received by the tissues at 5 cm for 5 minutes of treatment. The fluorescence had a peak wavelength of about 540-580 nm and a bandwidth of about 20-40 nm. The relative ratio of the power density of light (activating light to fluorescent light) received by the tissue varied during the treatment time.

### Example 2

### Protein Secretion and Gene Expression Profiles

Wounded and unwounded 3D human skin models (EpiDermFT, MatTek Corporation) were used assess the potential of a biophotonic material to trigger distinct protein secretion and gene expression profiles. Briefly, a biophotonic composition comprising Eosin and Erythrosine were placed on top of wounded and unwounded 3D human skin models cultured under different conditions (with growth factors, 50% growth factors and no growth factors). The skin models and the composition were separated by a nylon mesh of 20 micron pore size. Each skin model-composition combination was then irradiated with blue light (‘activating light’) for 5 minutes at a distance of 5 cm from the light source. The activating light consisted of light emitted from an LED lamp having an average peak wavelength of about 440-470 nm, a power density of 60-150 mW/cm² at 5 cm, and a total intensity after 5 minutes of about 18-39 J/cm². The controls were consisted of 3D skin models not illuminated with light.

Gene expression and protein secretion profiles were measured 24 hours post-light exposure. Cytokine secretion was analyzed by antibody arrays (RayBio Human Cytokine antibody array), gene expression was analyzed by PCR array (PAHS-013A, SABioscience) and cytotoxicity was determined by GAPDH and LDH release. Results (Tables 1 and 2) showed that the light treatment is capable of increasing the level of protein secreted and gene expression involved in the early inflammatory phase of wound healing in wounded skin inserts and in non-starvation conditions. In starvation conditions mimicking chronic wounds, there was no increase in the level of inflammatory protein secreted when compared to the control. Interestingly, the effect of the light treatment on unwounded skin models has a much lower impact at the cellular level than on wounded skin insert, which suggests an effect at the cellular effect level of the light treatment. It seems to accelerate the inflammatory phase of the wound healing process. Due to the lack of other cell types such as macrophages in the 3D skin model, the anti-inflammatory feed-back is absent and may explain the delay in wound closure. Cytoxicity was not observed in the light treatments.

TABLE 1
List of proteins with statistically significant difference
secretion ratio between treated and untreated control at
day 3. Two arrows mean that the ratio was over 2 folds.

| | Medium | Medium | Medium |
|---|---|---|---|
| | 1X | 0.5X | 0X |
| Increase | | ENA78 | Angiogenin |
| | | p = 0.04 ↑↑ | p = 0.03 ↑ |
| | | Il-1R4/ST2 | CXCL16 |
| | | p = 0.02 ↑↑ | p = 0.04 |
| | | MMP3 | ↑ |
| | | p = 0.01 ↑↑ | |
| | | MCP-2 | |
| | | p = 0.04 ↑↑ | |
| Decrease | BMP6 | BMP6 | |
| | p = 0.01 ↓ | p = 0.02 ↓ | |
| | TNFα | | |
| | p = 0.005 ↓ | | |

TABLE 2
List of genes with statistically significant difference expression
ratio between treated and untreated control during the first 24
hours. Two arrows mean that the ratio was over 2 folds

| | Medium | Medium | Medium |
|---|---|---|---|
| | 1X | 0.5X | 0X |
| Increase | CTGF | CTGF | MMP3 |
| | p = 0.02 ↑ | P = 0.04 ↑ | p = 0.007 ↑↑ |
| | ITGB3 | ITGB3 | LAMA1 |
| | p = 0.03 ↑ | p = 0.05 ↑ | p = 0.03 ↑ |
| | MMP1 | MMP1 | ITGA2 |
| | p = 0.03 ↑ | p = 0.02 ↑↑ | p = 0.03 ↑ |
| | MMP3 | MMP10 | |
| | p = 0.01 ↑ | p = 0.003 ↑↑ | |
| | THBS1 | MMP3 | |
| | P = 0.02 ↑ | p = 0.007 ↑↑ | |
| | | MMP8 | |
| | | p = 0.02 ↑↑ | |
| | | THBS1 | |
| | | p = 0.03 ↑ | |
| Decrease | HAS1 | NCAM1 | |
| | p = 0.009 ↓↓ | p = 0.02 ↓↓ | |
| | NCAM1 | VCAN | |
| | p = 0.05 ↓↓ | p = 0.02 ↓ | |
| | VCAM1 | LAMC1 | |
| | p = 0.03 ↓↓ | p = 0.002 ↓ | |
| | COL7A1 | COL6A1 | |
| | p = 0.04 ↓ | p = 0.007 ↓ | |
| | CTNNA1 | MMP7 | |
| | p = 0.03 ↓ | p = 0.003 ↓ | |

### Example 3

### Varying Power Density with Time and Distance

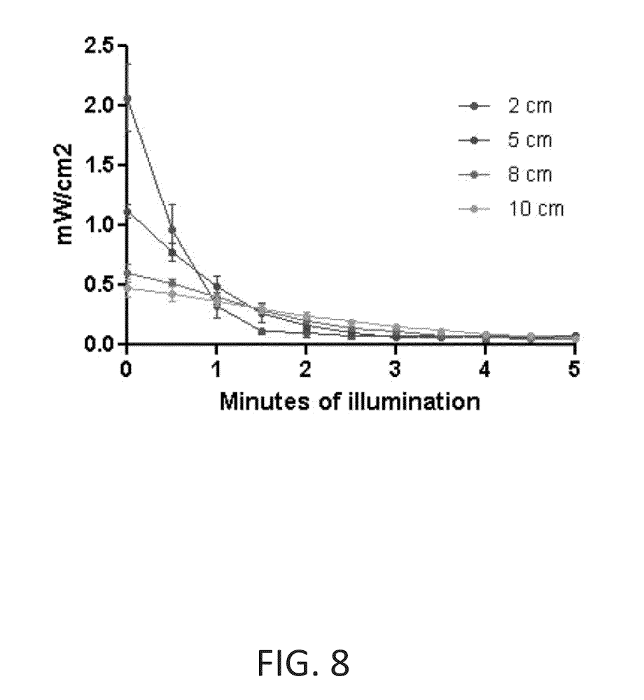

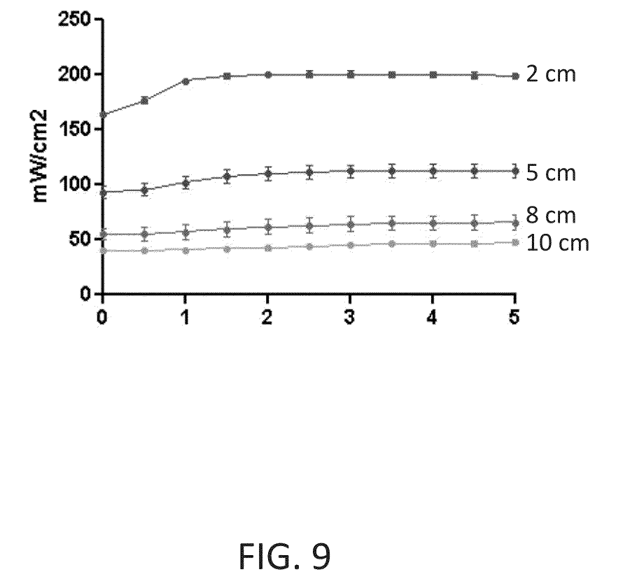

FIGS. 8 and 9 illustrate how an appropriate light treatment regimen may be selected for medical or cosmetic therapy according to embodiments of the present disclosure by varying the distance of the light source from the treatment site. FIG. 8 illustrates the decrease in fluorescence over a 5 minute treatment time. FIG. 9 illustrates an increase in an activating light, in this example, a blue light being activating and being transmitted through a biophotonic composition.

It is to be understood that the foregoing description is merely illustrative and is not to be limited to the details given herein. While several embodiments have been provided in the present disclosure, it should be understood that the disclosed systems, devices, and methods, and their components, may be embodied in many other specific forms without departing from the scope of the disclosure.

Variations and modifications will occur to those of skill in the art after reviewing this disclosure. The disclosed features may be implemented, in any combination and subcombinations (including multiple dependent combinations and subcombinations), with one or more other features described herein. The various features described or illustrated above, including any components thereof, may be combined or integrated in other systems. Moreover, certain features may be omitted or not implemented.

Examples of changes, substitutions, and alterations are ascertainable by one skilled in the art and could be made without departing from the scope of the information disclosed herein. All references cited herein are incorporated by reference in their entirety and made part of this application.

1. A device for phototherapy comprising:
a first light source which can emit a first light having an emission spectra for activating a photoactivatable composition applied on or near a treatment area; and
a second light source which can emit a second light having a different emission spectra from the first, wherein the first and the second emission spectra are in the blue and/or violet regions of the electromagnetic spectrum.

2. The device of claim 1, wherein the first light has a peak emission wavelength of about 430 to about 500 nm, about 440 to about 500 nm, about 450 to about 500 nm, about 430 to about 475 nm, about 435 nm to about 470 nm, about 440 nm, about 450 nm, about 460 nm or about 470 nm.

3. The device of claim 1, wherein the second light has a peak emission wavelength of about 400 nm to about 500 nm, about 400 nm to about 475 nm, about 400 nm to about 450 nm, about 400 nm to about 430 nm, or about 410 nm to about 420 nm, about 415 nm.

4. The device of claim 1, wherein the peak emission wavelength of the first light is from about 410 nm to about 430 nm, and the peak emission wavelength of the second light is from about 440 nm to about 470 nm.

5. The device of claim 1, wherein at least one of the first and second lights has a bandwidth of equal to or less than about 20 nm.

6. The device of claim 1, wherein at least one of the first and second lights has a bandwidth of about 19 nm±5 nm.

7. The device of claim 1, wherein an average power density of the light emitted by the device is about 10 to about 200 mW/cm², about 10 to about 150 mW/cm², 20 to about 130 mW/cm², about 55 to about 130 mW/cm², about 90 to about 140 mW/cm², about 100 to about 140 mW/cm², about 110 to about 135 mW/cm².

8. The device of claim 1, wherein an average power density of the light emitted by the device is about 10 to about 75 mW/cm², about 30 to about 70 mW/cm², about 40 mW/cm² to about 70 mW/cm², about 55 to about 65 mW/cm².

9. The device of claim 1, wherein the device is arranged to emit light having a fluence, during a single treatment, of more than about 4 J/cm², more than about 10 J/cm², more than about 15 J/cm², more than about 30 J/cm², more than about 50 J/cm², up to about 60 J/cm².

10. The device of claim 1, wherein the device is arranged to emit light, during a single treatment, having a fluence of about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², about 20 J/cm² to about 40 J/cm², or about 10 J/cm² to about 20 J/cm².

11. The device of claim 1, further comprising a controller for varying one or more of emission spectra parameters of the first and second lights, the emission spectra parameters being selected from bandwidth, peak wavelength, power density, time of emission and fluence.

12. The device of claim 11, wherein the controller can control separately one or more of the emission spectra parameters of the first and second lights.

13. The device of claim 11, wherein the controller is arranged to modulate one or more of the emission spectra parameters of the first and second lights as a function of treatment time.

14. The device of claim 1, further comprising a third light source, wherein the third light source can emit a third light having a peak wavelength of about 500 nm to about 750 nm, about 630 to about 750 nm.

15. The device of claim 1, wherein the first and second light sources can emit non-coherent light.

16. The device of claim 1, wherein the first and second light sources are light emitting diodes (LEDs).

17. The device of claim 16, wherein the LEDs are arranged as an array on at least one panel.

18. The device of claim 17, comprising a plurality of connectable panels.

19-25. (canceled)

26. A lamp, comprising
a lamp head having a plurality of light emitting diodes (LEDs) arranged in an array, the array comprising at least two sets of LEDs, wherein each set includes at least one LED;
a lamp controller electrically connected to the lamp head and having circuitry for controlling and operating the LEDs;
wherein the first set of LEDs can generate non-coherent light having a peak wavelength of about 430 nm to about 500 nm;
wherein the second set of LEDs can generate non-coherent light having a peak wavelength of about 400 nm to about 430 nm;
wherein a power density of light which can be generated by the lamp head is from about 10 to about 75 mW/cm², or from about 55 mW/cm² to about 150 mW/cm².

27. A lamp according to claim 26, wherein the first set of LEDs can generate light having a full width half maximum bandwidth of about 19 nm±5 nm, about 13 to about 26 nm.

28. A lamp according to claim 26, wherein the second set of LEDs can generate light having a full width half maximum bandwidth of about 13 nm to about 20 nm.

29. The lamp of claim 26, further comprising a third set of LEDs, wherein the third set of LEDs generates non-coherent light having a peak wavelength of about 500 nm to 750 nm.

30. The lamp of claim 26, wherein the controller is arranged to vary one or more parameters of the of light emitted from the first and second light sources, the one or more parameters being selected from power density, bandwidth, wavelength, fluence and emission time.

31-40. (canceled)

41. A method for cosmetic or medical treatment of tissue, said method comprising:
irradiating said tissue with a first light having a first emission spectra which can activate a photoactivatable composition applied on or near a treatment area; and
a second light having a second emission spectra, different from the first emission spectra, wherein the first and the second emission spectra have peak wavelengths in the blue and/or violet regions of the electromagnetic spectrum.

42. The method of claim 41, wherein the first light has a peak emission wavelength of about 430 to about 500 nm, about 440 to about 500 nm, about 450 to about 500 nm, about 430 to about 475 nm, about 435 nm to about 470 nm, about 440 nm, about 450 nm, about 460 nm or about 470 nm.

43. The method of claim 40, wherein the second light has a peak emission wavelength of about 400 nm to about 500 nm, about 400 nm to about 475 nm, about 400 nm to about 450 nm, about 400 nm to about 430 nm, or about 410 nm to about 420 nm, about 415 nm.

44. The method of claim 40, wherein the peak emission wavelength of the first light is from about 410 nm to about 430 nm, and the peak emission wavelength of the second light is from about 440 nm to about 470 nm.

45. The method of claim 40, wherein at least one of the first and second lights has a bandwidth of equal to or less than about 20 nm.

46. The method of claim 40, wherein at least one of the first and second lights has a bandwidth of about 19 nm±5 nm.

47. The method of claim 40, wherein an average power density of the light irradiating the skin is about 10 to about 200 mW/cm², 10 to about 150 mW/cm², 20 to about 130 mW/cm², about 55 to about 130 mW/cm², about 90 to about 140 mW/cm², about 100 to about 140 mW/cm², about 110 to about 135 mW/cm².

48. The method of claim 40, wherein an average power density of the light irradiating the skin, is about 10 to about 75 mW/cm², about 20 to about 70 mW/cm², about 30 mW/cm² to about 70 mW/cm², about 45 to about 65 mW/cm².

49. The method of claim 40, wherein a light intensity on the skin during a single treatment is more than about 4 J/cm², more than about 10 J/cm², more than about 15 J/cm², more than about 30 J/cm², more than about 50 J/cm², up to about 60 J/cm².

50. The method of claim 40, wherein a light intensity on the skin during a single treatment is about 4 J/cm² to about 60 J/cm², about 10 J/cm² to about 60 J/cm², about 10 J/cm² to about 50 J/cm², about 10 J/cm² to about 40 J/cm², about 10 J/cm² to about 30 J/cm², or about 10 J/cm² to about 20 J/cm².

51. The method of claim 40, the method further comprising modulating with treatment time at least one of peak wavelength, wavelength range and power density of the first and/or second lights.

52. The method of claim 40, wherein the first and/or second lights are generated by light emitting diodes (LEDs).

53-57. (canceled)

58. A method for cosmetic or medical treatment of tissue, said method comprising:
irradiating the tissue with non-coherent light from a first light source having a peak wavelength of about 430 nm to about 500 nm;
irradiating the tissue with non-coherent light from a second light source having a peak wavelength of about 400 nm to about 430 nm; wherein the tissue is irradiated with a total power density of light of about 10 to about 75 mW/cm², or about 55 mW/cm² to about 150 mW/cm².

59. The method of claim 58, further comprising applying a photoactivatable composition to the tissue prior to irradiating with the light.

60-61. (canceled)
